(19) United States
(12) Patent Application Publication
(10) Pub. No.: US 2017/0055935 A1
(43) Pub. Date: Mar. 2, 2017

(54) MEDICAL IMAGE SCANNING APPARATUS AND MEDICAL IMAGE SCANNING METHOD

(71) Applicant: HITACHI, LTD., Tokyo (JP)

(72) Inventor: Kentaro TAKAHASHI, Tokyo (JP)

(73) Assignee: Hitachi, Ltd., Tokyo (JP)

(21) Appl. No.: 15/303,147

(22) PCT Filed: Mar. 25, 2015
(86) PCT No.: PCT/JP2015/059030

§ 371 Date: Oct. 10, 2016

(30) Foreign Application Priority Data
Apr. 11, 2014 (JP) 2014-081780

(51) Int. Cl. A61B 6/00; A61B 5/00; A61B 6/03; A61B 5/055

(52) U.S. Cl. CPC A61B 6/545; A61B 5/055; A61B 5/742; A61B 6/032; A61B 6/463; A61B 6/4435; A61B 6/467

(57) ABSTRACT

In order to reduce a burden on an operator for setting scanning conditions by configuring so that an index value can be estimated immediately by setting a parameter and the value from among scanning conditions, the present invention provides a medical image scanning apparatus that acquires and displays tomographic images of an object and is characterized by comprising: an index value calculation unit that calculates an index value based on each parameter value of scanning conditions; a scale setting unit that sets a scale of each parameter axis according to the calculated index value; and a display control unit that displays a relational diagram including a graphic indicating the magnitude of the calculated index value and each parameter axis having the set scale.

### CROSS-REFERENCE TO RELATED APPLICATIONS

This application is a national phase claiming the benefit of and priority to International Patent Application No. PCT/JP2015/059030, entitled “MEDICAL IMAGING DEVICE AND MEDICAL IMAGING METHOD”, filed Mar. 25, 2015, which claims priority to Japanese Patent Application No. 2014-081780, entitled “MEDICAL IMAGING DEVICE AND MEDICAL IMAGING METHOD”, filed Apr. 11, 2014, which are hereby incorporated by reference in their entireties.

### TECHNICAL FIELD

The present invention relates to a medical image scanning apparatus such as an X-ray CT (Computed Tomography) apparatus, an MRI (Magnetic Resonance Imaging) apparatus, or the like, and, in particular, to a technique to support scanning condition setting.

### BACKGROUND ART

A medical image scanning apparatus typified by an X-ray CT apparatus is an apparatus for imaging an internal body of an object and is used for diagnosing such as finding a lesion. Scanning conditions of the medical image scanning apparatus include various parameters such as a tube voltage and a tube current of an X-ray tube, a rotational speed of a scanner, and a bed moving speed in a case of an X-ray CT apparatus, for example. While setting the scanning conditions based on image quality to be acquired, an operator needs to pay attention to an index value other than image quality, such as an X-ray exposure dose.

Patent Literature 1 discloses that an exposure dose under the set scanning conditions is displayed on a two-dimensional map of a tube voltage and a tube current.

### CITATION LIST

### Patent Literature

PTL 1: Japanese Unexamined Patent Publication No. 2013-215473

### SUMMARY OF INVENTION

### Technical Problem

However, in PTL 1, although an exposure dose in the set scanning conditions can be checked, this does not allow an operator to check a parameter and the value to be set from among the scanning conditions in order to set the exposure dose to equal to or less than a specified value. Also, in order to set an index value other than the exposure dose, such as an SAR (Specific Absorption Ratio) of an MRI apparatus, to equal to or less than a specified value (upper limit value), there is a need to immediately check a parameter and the value that should be changed from among the scanning conditions.

Therefore, the present invention has a purpose to provide a medical image scanning apparatus capable of reducing a burden on an operator for setting scanning conditions.

### Solution to Problem

In order to achieve the above purpose, the present invention is characterized by setting a scale of each parameter axis according to an index value calculated based on each parameter value of scanning conditions and displaying a relational diagram comprising a graphic indicating the magnitude of a calculated index value and each parameter axis having a set scale.

Specifically, the present invention is a medical image scanning apparatus that acquires and displays tomographic images of an object and is characterized by comprising an index value calculation unit that calculates an index value based on each parameter value of scanning conditions, a scale setting unit that sets a scale of each parameter axis according to the calculated index value, and a display control unit that displays a relational diagram including a graphic indicating the magnitude of the calculated index value and each parameter axis having a set scale.

### Advantageous Effects of Invention

According to the present invention, a medical image scanning apparatus capable of reducing a burden on an operator for setting scanning conditions can be provided.

### BRIEF DESCRIPTION OF DRAWINGS

FIG. 1 is an overall configuration diagram of an X-ray CT apparatus of the present invention.

FIG. 2 is a diagram showing a functional configuration of a first embodiment.

FIG. 3 is a diagram showing a process flow of the first embodiment.

FIG. 4 is a diagram showing an example of a scanning condition setting window.

FIG. 5 is a diagram showing an example of a relational diagram between an exposure dose that is an example of an index value and each parameter.

FIG. 6 is a diagram showing a relational diagram when a tube voltage is changed from the relational diagram of FIG. 5.

FIG. 7 is a diagram showing a relational diagram when a tube current is changed from the relational diagram of FIG. 6.

FIG. 8 is a diagram showing an example of a relational diagram between an exposure dose, an image SD, and each parameter.

FIG. 9 is a diagram showing the other example of a relational diagram between one index value and each parameter (a second embodiment).

FIG. 10 is a diagram showing a relational diagram when a tube voltage is changed from the relational diagram of FIG. 9.

FIG. 11 is a diagram showing a window example of displaying a relational diagram between one index value and each parameter along the body-axis direction.

FIG. 12 is a diagram showing the other example of a relational diagram between one index value and each parameter (a third embodiment).

FIG. 13 is a diagram showing a relational diagram when a tube current axis is added to the relational diagram of FIG. 12.

FIG. 14 is an overall configuration diagram of an MRI apparatus of the present invention (a fourth embodiment).

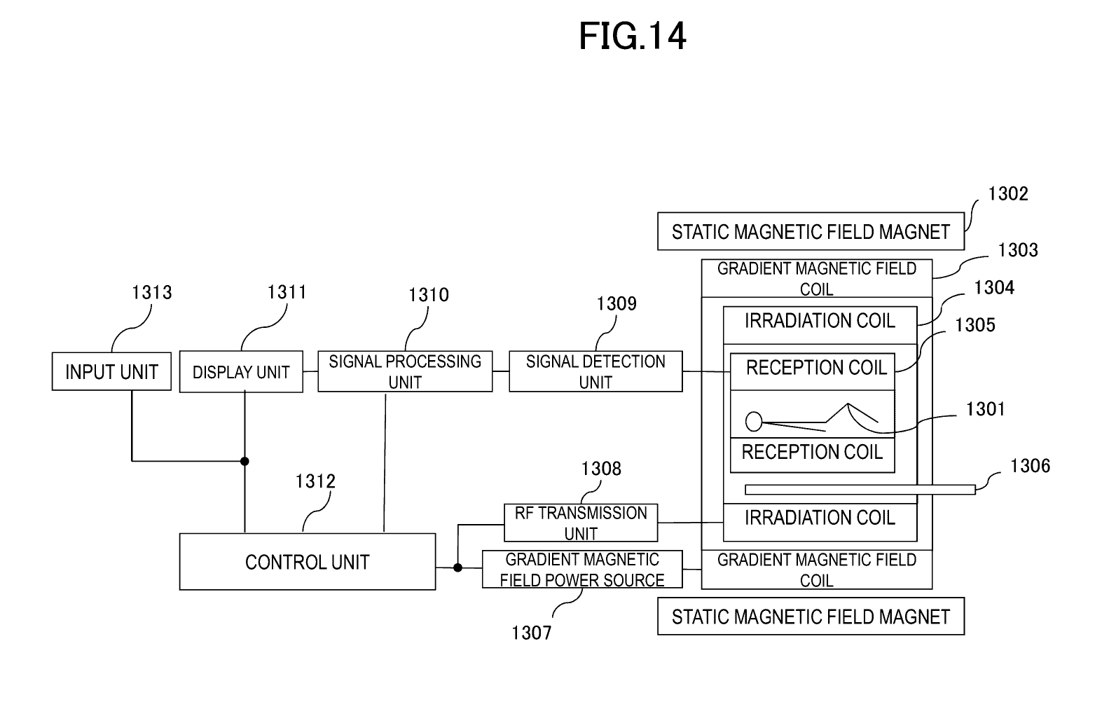

FIG. 15 is a diagram showing an example of a relational diagram between a SAR that is an example of an index value and each parameter.

### DESCRIPTION OF EMBODIMENTS

Hereinafter, desirable embodiments of the medical image scanning apparatus related to the present invention will be described according to the attached diagrams. It is noted that the same reference signs are provided for components having the same functional configurations to omit repeated description in the following description and the attached diagrams.

### First Embodiment

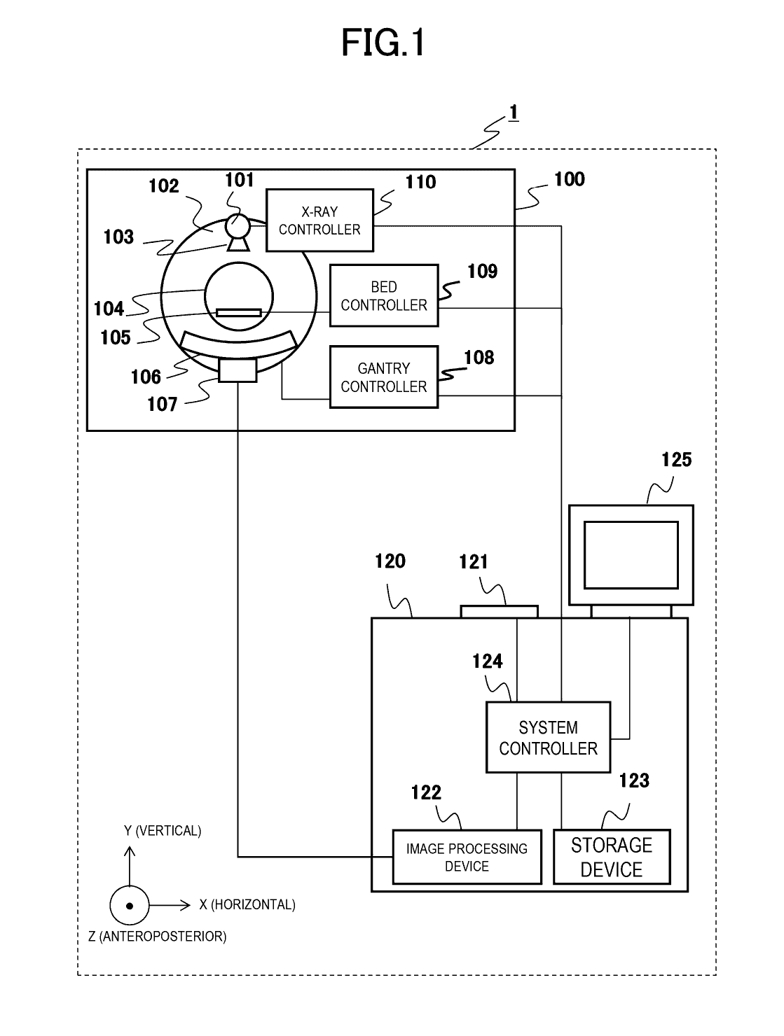

FIG. 1 is a block diagram showing an overall configuration of an X-ray CT apparatus that is an example of a medical image scanning apparatus. An X-ray CT apparatus 1 includes a scan gantry unit 100 and an operation unit 120 as shown in FIG. 1.

The scan gantry unit 100 comprises an X-ray tube device 101, a rotating disk 102, a collimator 103, an X-ray detector 106, a data acquisition system 107, a bed device 105, a gantry controller 108, a bed controller 109, and an X-ray controller 110.

The X-ray tube device 101 is a device that irradiates an X-ray to an object placed on a bed device 105. The collimator 103 is a device that limits an irradiation range of an X-ray to be irradiated from the X-ray tube device 101. The rotating disk 102 is provided with an opening 104 to accommodate an object placed on the bed device 105, includes the X-ray tube device 101 and the X-ray detector 106, and rotates around the object.

The X-ray detector 106 is disposed opposite to the X-ray tube device 101 and measures a spatial distribution of transmitted X-rays by detecting X-rays transmitted through an object, in which a number of detection elements are one-dimensionally arranged in a rotation direction of the rotating disk 102 or a number of detection elements are two-dimensionally arranged in the rotation direction and a rotation-axis direction of the rotating disk 102. The data acquisition system 107 is a device that acquires an X-ray amount detected by the X-ray detector 106 as digital data.

The gantry controller 108 is a device that controls rotation and inclination of the rotating disk 102. The bed controller 109 is a device that controls vertical, anteroposterior, and horizontal movements of the bed device 105. The vertical, anteroposterior, and horizontal directions are illustrated in FIG. 1 and are respectively referred to also as Y, Z, and X directions in the subsequent description. The X-ray controller 110 is a device that controls electric power to be input in the X-ray tube device 101.

The operation unit 120 comprises an input device 121, an image processing device 122, a display device 125, a storage device 123, and a system controller 124. The input device 121 is a device for inputting an object name, an examination date, scanning conditions, and the like and is, specifically, a keyboard, a pointing device, a touch panel, or the like. The image processing device 122 is a device that reconstructs a CT image by performing calculation processing for measurement data to be sent out of the data acquisition system 107.

The display device 125 is a device that displays a CT image or the like generated in the image processing device 122 and is, specifically, a CRT (Cathode-Ray Tube), a liquid-crystal display, or the like. The storage device 123 is a device that stores data acquired by the data acquisition system 107, image data of CT images generated in the image processing device 122, and the like and is, specifically, an HDD (Hard Disk Drive) or the like. The system controller 124 is a device that controls these devices, the gantry controller 108, the bed controller 109, and the X-ray controller 110. Also, the system controller 124 may execute a process flow to be described later.

The X-ray tube device 101 irradiates an X-ray to an object according to scanning conditions by controlling electric power to be input to the X-ray tube device 101 by the X-ray controller 110 based on the scanning conditions in particular, such as an X-ray tube voltage and an X-ray tube current, input from the input device 121. The X-ray detector 106 detects an X-ray irradiated from the X-ray tube device 101 and transmitted through an object using a number of X-ray detection elements and measures a distribution of the transmitted X-ray. The rotating disk 102 is controlled by the gantry controller 108 and rotates based on the scanning conditions in particular, such as a rotational speed, input from the input device 121. The bed device 105 is controlled by the bed controller 109 and operates based on the scanning conditions in particular, such as a helical pitch, input from the input device 121.

X-ray irradiation from the X-ray tube device 101 and measurement of transmitted X-ray distribution by the X-ray detector 106 are repeated with rotation of the rotating disk 102, which acquires projection data from various angles. The projection data is associated with a view (View) representing each angle, a channel (ch) number that is a detection element number of the X-ray detector 106, and a column number. The projection data acquired from various angles is transmitted to the image processing device 122. The image processing device 122 performs a back projection process for the transmitted projection data from various angles in order to reconstruct a CT image. The CT image acquired by reconstruction is displayed on the display device 125.

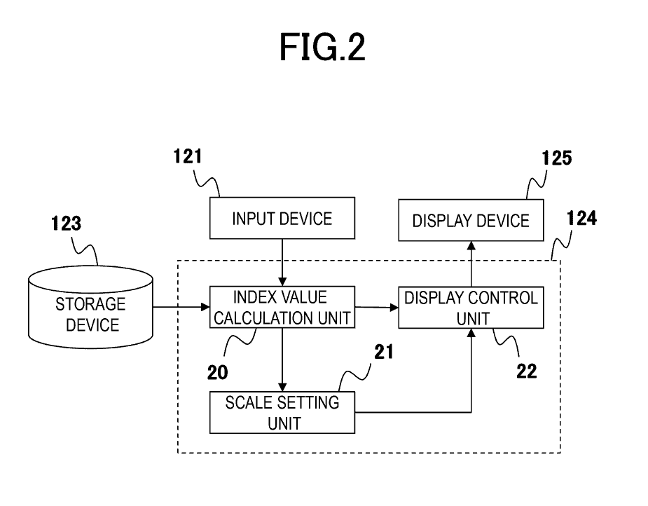

Using FIG. 2, a functional configuration of the X-ray CT apparatus 1 of the present embodiment will be described. It is noted that the functional configuration may be configured by exclusive hardware or by software operating on the system controller 124. Here, a case of configuration by software will be described.

The system controller 124 of the X-ray CT apparatus 1 is provided with an index value calculation unit 20, a scale setting unit 21, and a display control unit 22. Hereinafter, each configuration unit will be described.

The index value calculation unit 20 calculates an index value based on each parameter value of scanning conditions. In order to calculate an index value, a relational expression showing a relationship between each parameter set in advance and the index value may be used, or a corresponding table showing correspondence between each parameter stored in the storage device 123 and the index value may be used. Index values of the present embodiment include, for example, an exposure dose of an object, an image SD (Standard Deviation) showing a noise amount of CT images to be acquired. A value input through the input device 121 or a value previously stored in the storage device 123 is used for each parameter value.

The scale setting unit 21 sets a scale for each parameter axis according to an index value calculated by the index value calculation unit 20. The scale for each parameter axis is set so as to correspond to a unit amount of the index value in a case where parameters other than the said parameter are fixed. For example, in a case where the index value is proportional to an A-fold parameter, the scale of the parameter axis is equivalent to 1/A of the index value, and in a case where the index value is proportional to a square parameter, the scale of the parameter axis is equivalent to a square root of the index value.

On the display device 125, the display control unit 22 displays a relational diagram comprising a graphic indicating the magnitude of a calculated index value by the index value calculation unit 20 and each parameter axis having a scale corresponding to a unit amount of the index value. For example, a marker on the axis, a bar graph, or a plane diagram may be used for the diagram showing the magnitude of a calculated index value. The relational diagram to be displayed on the display device will be described in detail later.

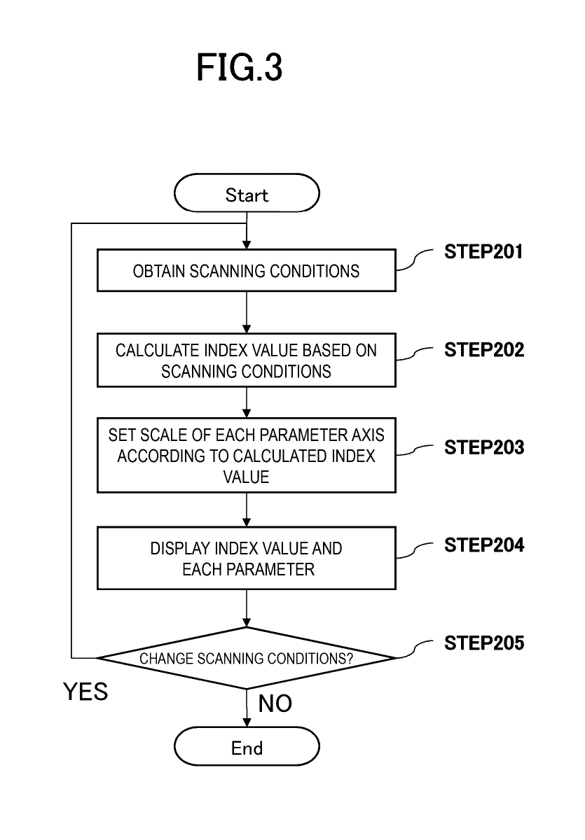

Using FIG. 3, a process flow in the present embodiment will be described.

(Step 201)

The index value calculation unit 20 obtains scanning conditions. Specifically, each parameter value input through the input device 121 is received, or each parameter value stored in the storage device 123 is read out.

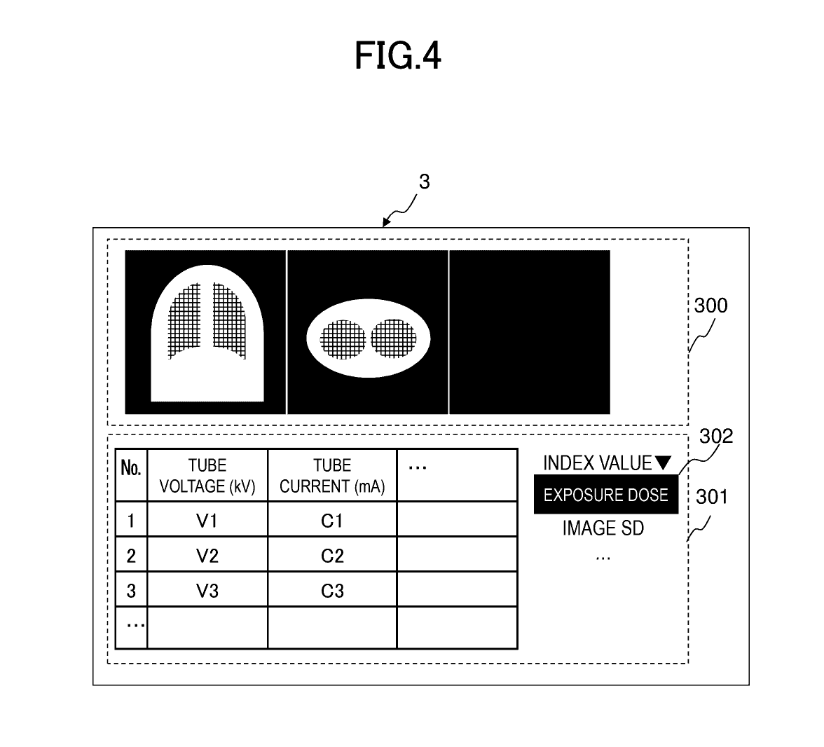

FIG. 4 shows an example of a scanning condition setting window. A window 3 comprises an image display area 300 and a scanning condition display area 301. The image display area 300 displays a scanned image. In the present embodiment, the image display area 300 is not always necessary. The scanning condition display area 301 displays scanning conditions for each scan number such as a tube voltage and a tube current of the X-ray tube device 101 and each parameter such as a rotational speed of the rotating disk 102. Also, an index value selecting part 302 may be provided for selecting a desired index value from among a plurality of index values. In the window 3, the index value selecting part 302 comprises a pull-down menu, in which an exposure dose is selected.

(Step 202)

The index value calculation unit 20 calculates an index value based on scanning conditions obtained in Step 201. In order to calculate an index value, a preset relational expression or a corresponding table stored in the storage device 123 may be used. In the subsequent description, the following relational expression will be used.

D=f(V,C,t,p) (1)

It is noted that the components of the formula are as follows: D: exposure dose, V: tube voltage, C: tube current, t: scan time, p: helical pitch, and f( ) a relational expression showing a relationship between the exposure dose, the tube voltage, the tube current, the scan time, and the helical pitch.

Here, in a case of setting V4, C4, t3, and p4 respectively for parameters of scanning conditions, i.e. a tube voltage, a tube current, a scan time, and a helical pitch, an exposure dose D is calculated as f(V4, C4, t3, p4) by using the formula (1).

(Step 203)

The scale setting unit 21 sets a scale of each parameter axis according to an index value calculated in Step 202. For example, in a case where an index value, i.e. an exposure dose is f(V4, C4, t3, p4), a scale of the tube voltage is associated with the exposure dose and set so that the relationship D=f(V, C4, t3, p4) is achieved. That is, while the parameters other than the tube voltage, i.e. the tube current, the scan time, and the helical pitch are still set to C4, t3, and p4 respectively, the scale of the tube voltage is set by associating with an exposure dose value, i.e. an index value. Similarly, in a case where an exposure dose is f(V4, C4, t3, p4), the parameters are associated with the exposure dose and set so that a relationship D=f(V4, C, t3, p4) for a scale of the tube current, a relationship D=f(V4, C4, t, p4) for a scale of the scan time, and a relationship D=f(V4, C4, t3, p) for a scale of the helical pitch are respectively achieved.

(Step 204)

On the display device 125, the display control unit 22 displays a relational diagram showing a relationship between an index value and each parameter based on an index value calculated in Step 202 and a scale of each parameter axis set in Step 203.

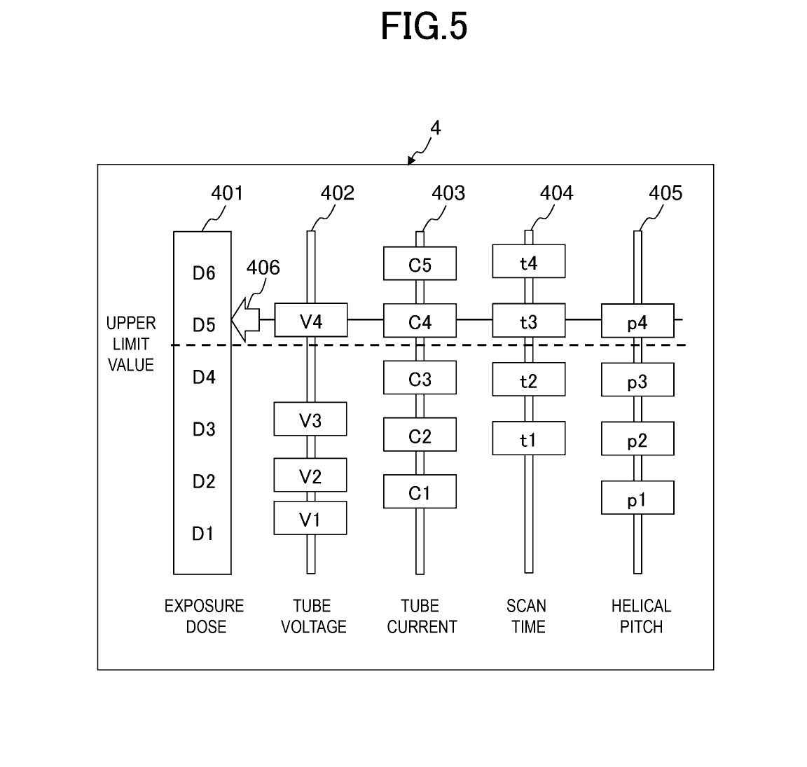

FIG. 5 shows an example of the relational diagram. A relational diagram 4 includes axes 401 to 405 and a marker 406. The axes 401 to 405 represent an exposure dose, a tube voltage, a tube current, a scan time, and a helical pitch respectively and are arranged parallel to each other. Also, scales of the axes 402 to 405 are respectively associated with the axis 401 of an index value. That is, when one parameter is changed, it is associated so as to check how the index value changes.

The marker 406 is a figure indicating the magnitude of an index value calculated based on set scanning conditions. In the relational diagram 4 of FIG. 5, the tip of the marker 406 indicates the magnitude of an index value in a case where V4, C4, t3, and p4 are set respectively for a tube voltage, a tube current, a scan time, and a helical pitch on the axis 401. That is, it is shown that an exposure dose is D5. Also, instead of the marker 406, the relational diagram 4 may be configured using a bar graph indicating the magnitude of an index value.

Also, a figure showing an upper limit value of an exposure dose may be displayed as a reference value of the exposure dose that is an index value. In the relational diagram 4, the upper limit value of the exposure dose is indicated by a broken line between D4 and D5. Displaying such a reference value helps an operator to determine whether or not the index value calculated based on scanning conditions is appropriate. In the relational diagram 4 of FIG. 5, it can be determined that the exposure dose exceeds the upper limit value under the set scanning conditions.

(Step 205)

The system controller 124 determines whether or not scanning conditions are changed. When the scanning conditions are changed, the procedure goes back to Step 201, and when the scanning conditions are not changed, the procedure ends here. Whether or not the scanning conditions are changed is determined by whether or not parameter values are newly set.

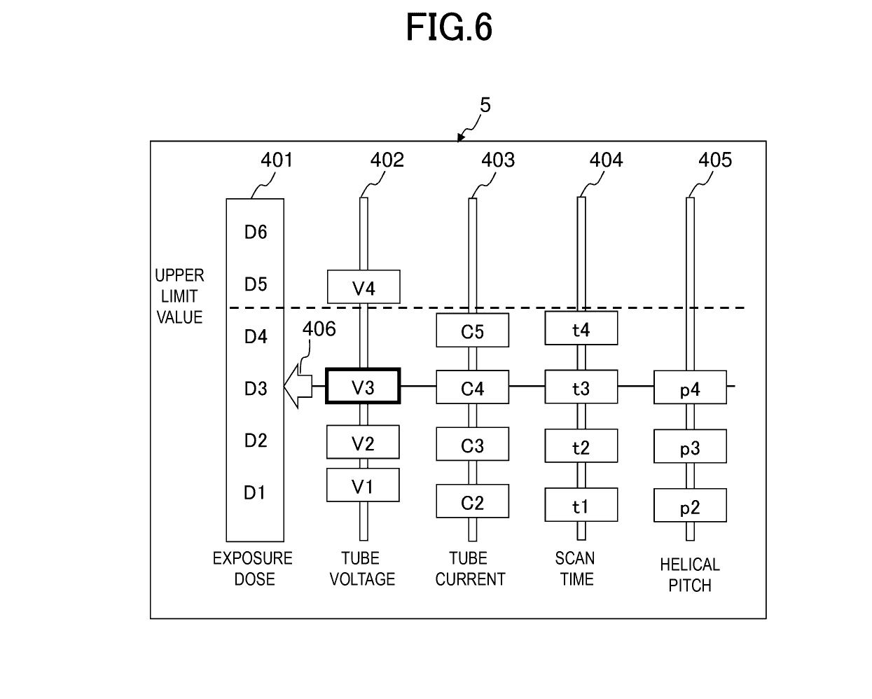

For example, a relational diagram 5 of FIG. 6 shows a state where a tube voltage value is changed from V4 to V3 in the relational diagram 4 of FIG. 5 and is a result after determining by the system controller 124 that scanning conditions are changed, going back to Step 201, and executing processes of Steps 201 to 204. Hereinafter, each step will be described.

In Step 201, the index value calculation unit 20 obtains a changed scanning condition, i.e. a tube voltage value V3. In Step 202, the index value calculation unit 20 calculates an index value according to the changed scanning condition. Here, an exposure dose is calculated as a D3 value because the tube voltage value was set to V3. In Step 203, the scale setting unit 21 sets a scale of each parameter axis according to the calculated exposure dose. Here, a tube voltage axis 402 is not changed because the exposure dose is changed from D5 to D3 according to the change of the tube voltage value, and scales of the axes 403 to 405 other than the tube voltage are changed according to the exposure dose value. In Step 204, the display control unit 22 displays the relational diagram 5 of FIG. 6 based on the process results of Steps 202 and 203 on the display device 125.

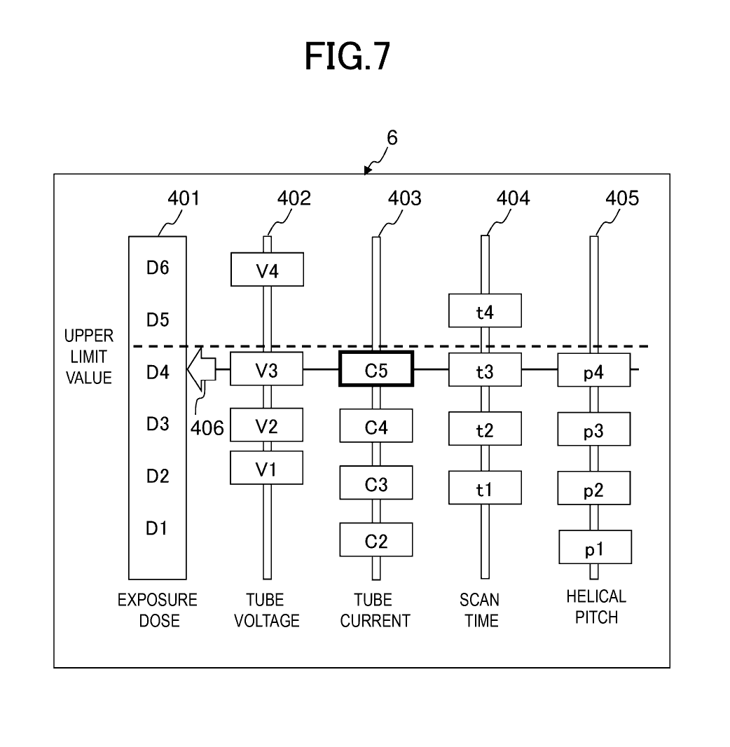

Also, a relational diagram 6 of FIG. 7 shows a state where a tube current value is changed from C4 to C5 in the relational diagram 5 of FIG. 6 and is a result after executing processes of Steps 201 to 204 by the system controller 124. Hereinafter, each step will be described. In the relational diagram 6 of FIG. 7, a tube current axis 403 is not changed from FIG. 6, and scales of the axes 402, 404, and 405 other than the tube current are changed according to the exposure dose value.

Also, in a case where an index value is an exposure dose, a past scanning history of an object is obtained to calculate an exposure history from the obtained scanning history, and the calculated exposure history may be accumulated to the exposure dose of this-time scanning and displayed. By accumulating and displaying the exposure history, an operator is allowed to set scanning condition while considering the scanning history.

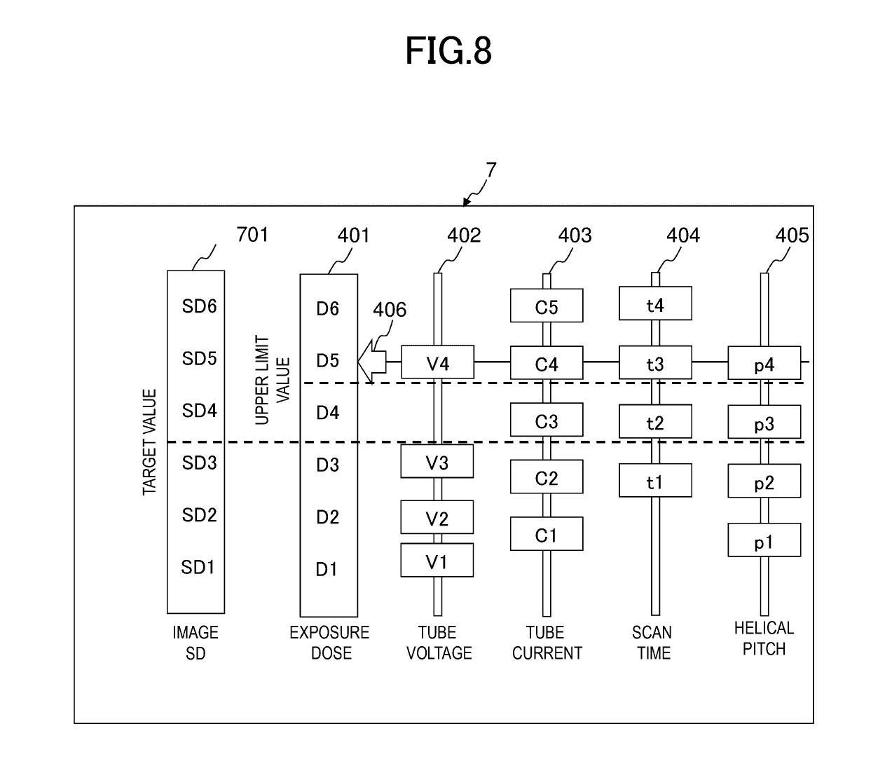

Although only the exposure dose is displayed as an index value in the relational diagrams 4 to 6 of FIGS. 5 to 7, a plurality of index values may be displayed on a relational diagram. FIG. 8 is an example relational diagram displaying a plurality of index values, on which an image SD axis 701 is displayed next to the left of an exposure dose axis 401.

In a case of displaying a plurality of index values on a relational diagram, one value of a plurality of the index values is calculated by the index value calculation unit 20 based on a set scanning condition. Then, the scale setting unit 21 sets a scale of each parameter axis according to the one calculated index value, and a scale of the other index value axis is set according to the scale of each parameter axis. In a relational diagram 7 of FIG. 8, an exposure dose is calculated as D5 based on the tube voltage V4, the tube current C4, the scan time t3, and the helical pitch p4 that were set as scanning conditions, and scales of the parameter axes 402 to 405 are set respectively according to the exposure dose value. Then, a scale of the image SD axis 701 is set according to the scales of the parameter axes 402 to 405. On the image SD axis 701, a broken line showing a target value of a preset image SD may be displayed as a reference value of the image SD. It is noted that the marker 406 indicates that the exposure dose and the image SD are D5 and SD5 respectively.

Also, although an exposure dose and an image SD were described as an example of an index value, the index value is not limited to these. For example, a time required for examination, i.e. a time from starting scanning to generating a diagnostic medical image may be set as an index value. In a case where the time required for examination is set as an index value, a relational diagram for setting a scan time, the number of scans, the number of reconstructed images, a level of successive approximation processing, a delay time and the like may be displayed as parameters of scanning conditions.

According to the embodiment described above, an index value can be estimated immediately by setting a parameter and the value from among scanning conditions, which can reduce a burden on an operator for setting scanning conditions.

### Second Embodiment

Next, a second embodiment will be described. In the first embodiment, an index value axis is arranged parallel to each parameter axis in the relational diagrams. In the present embodiment, each parameter axis is arranged radially to display the magnitudes of index values using an area of a plane figure. That is, the present embodiment is different from the first embodiment in configuration of a relational diagram showing a relationship between the index values and each parameter, and the other components are similar to the first embodiment. Description of the similar components will be omitted.

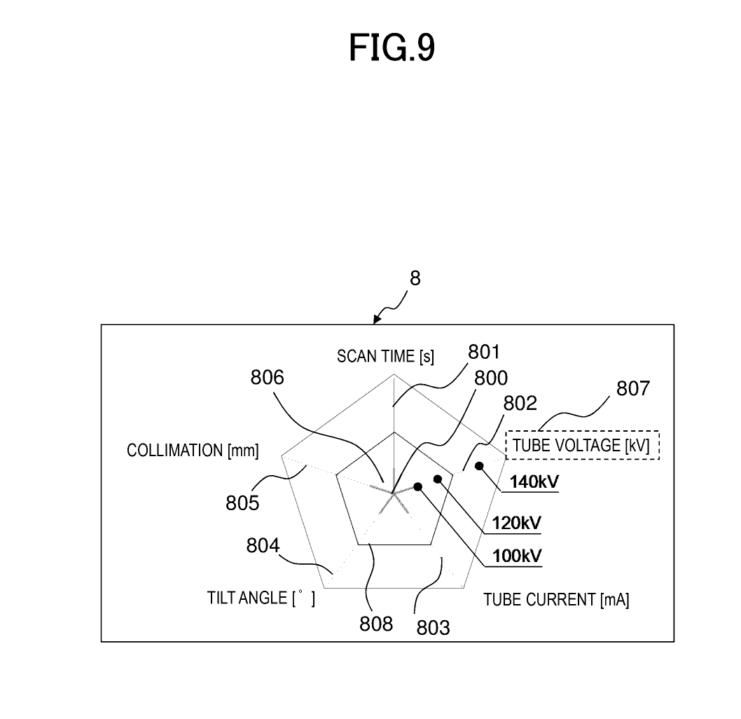

FIG. 9 shows an example of the relational diagram of the present embodiment. A relational diagram 8 includes axes 801 to 805 and a plane FIG. 806. The axes 801 to 805 show a scan time, a tube voltage, a tube current, a tilt angle that is an inclination angle of the rotating disk 102, and a collimation that is an X-ray irradiation width and are arranged radially from an origin 800 at equal angles around the origin 800. The axes 801 to 805 correspond to the magnitudes of index values index values respectively. That is, similarly to the first embodiment, the index values are calculated based on scanning conditions, and scales of the axes 801 to 805 are set so that distances from the origin 800 represent the magnitudes of the calculated index values.

The plane FIG. 806 is a figure indicating the magnitudes of index values using an area. Points corresponding to the magnitudes of the index values are calculated on each axis, and the plane FIG. 806 is formed by connecting the calculated points. That is, the plane FIG. 806 is a regular polygon centered at the origin 800 and has vertices whose number is the same as the parameter axes. In the relational diagram 8 of FIG. 9, the plane FIG. 806 is a regular pentagon with five parameter axes. Because a scale of each parameter axis is set so that distances from the origin 800 represent the magnitudes of the index values, a shape of the plane FIG. 806 may be a circle, a sector, or a polygon formed by connecting arbitrary points and end points on the arc of the sector. It is noted that the calculated index values may be displayed in numerical values with the plane FIG. 806.

Because an area of the plane FIG. 806 represents the magnitudes of index values, scales of the axes 801 to 805 may be set so as to correspond to square roots of the index values. When the scales of the axes 801 to 805 are set so as to correspond to the square roots of the index values, an area of the plane FIG. 806 is changed in proportion to the magnitudes of the index values, which makes easy for an operator to intuitively check the magnitudes of the index values.

Also, a figure showing target values of index values may be displayed as reference values on a relational diagram. In the relational diagram 8 of FIG. 9, the target values of the index values are displayed using a regular polygon 808. Displaying such target values helps an operator to determine whether or not the index values calculated based on scanning conditions are appropriate. In the relational diagram 8 of FIG. 9, it can be determined that the index values do not reach the target values under the set scanning conditions.

When an operator checks the relational diagram 8 of FIG. 9 and thinks that a scanning condition such as a tube voltage should be changed, the operator specifies an indication label 807 of the tube voltage. When the indication label 807 of the tube voltage is specified, values that can be selected as the tube voltage are displayed on an axis 802. In the relational diagram 8 of FIG. 9, 100 kV, 120 kV, and 140 kV are displayed as the values that can be selected.

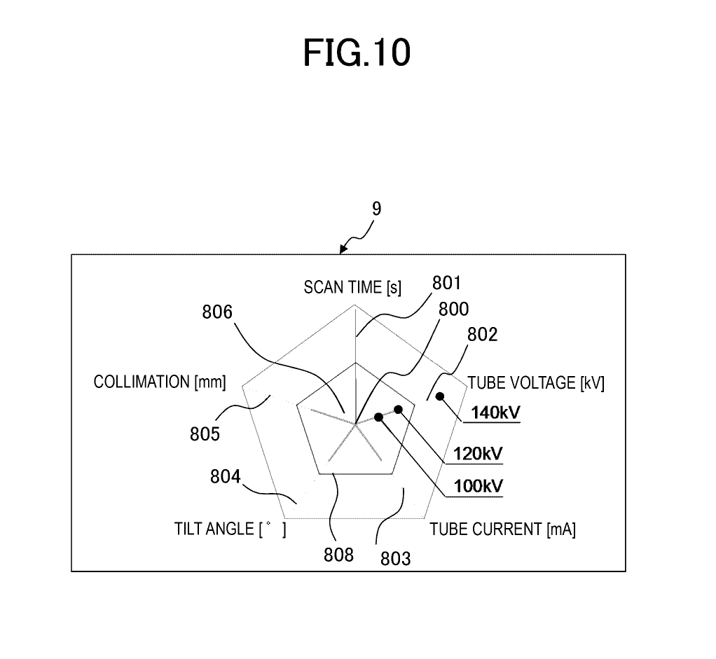

Furthermore, when 120 kV is selected as a tube voltage value in the relational diagram 8 of FIG. 9, the display is switched to the relational diagram 9 of FIG. 10. That is, similarly to the first embodiment, an index value is calculated based on the changed scanning condition, and the magnitude of the calculated index value is reflected to an area of the plane FIG. 806. At this time, parameters other than the tube voltage are not changed, scales of the axes 801 and 803 to 805 of the parameters other than the tube voltage are changed according to the magnitude of the index value.

For example, 200 mA is indicated at the intersection of the axis 803 of the tube current and the plane FIG. 806 in a case where a tube current is set to 200 mA in the relational diagram 8 of FIG. 9. Hence, the scale of the axis 803 of the tube current is changed so that 200 mA is indicated at the intersection of the axis 803 of the tube current and the plane FIG. 806 also in the relational diagram 9 of FIG. 10.

Also, when one parameter in scanning conditions is changed in the present embodiment, it may be configured so that the peaks of the plane FIG. 806, i.e. the intersections between the axes 801 and 805 of each parameter are moved by mouse operation on the axes 801 and 805.

Although the configuration of the relational diagram of the present embodiment is displayed like what is called a radar chart, the scales of the axes 801 to 805 of each parameter are, differently from the radar chart, set according to the magnitude of a calculated index value, i.e. the same scale. Also, a shape of the plane FIG. 806 that represents the magnitudes of index values is always a regular polygon or a circle, which makes easy to check the magnitudes of the index values with an area of the plane FIG. 806.

According to the embodiment described above, an index value can be estimated immediately by setting a parameter and the value from among scanning conditions, which can reduce a burden on an operator for setting scanning conditions. Also, according to the relational diagrams of the present embodiment, a display area can be reduced more than the relational diagram of the first embodiment, which is advantageous to configuring a window that requires a display of the other information.

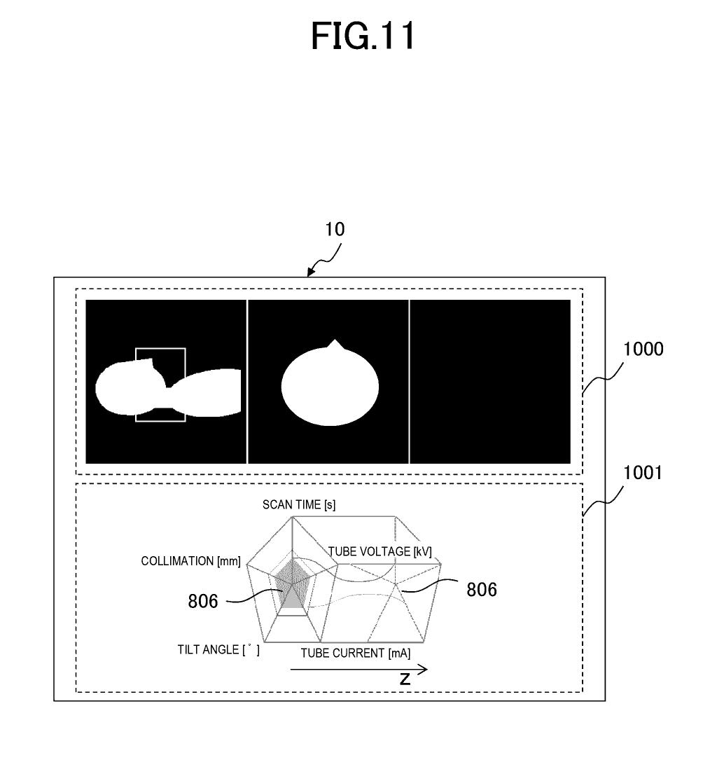

For example, in a case where scanning conditions differ depending on an object's position in the body-axis direction, a window 10 may be displayed as illustrated in FIG. 11. The window 10 is provided with an image display area 1000 and a scanning condition display area 1001. The image display area 1000 displays a scanned image such as a scanogram image. The object's position in the body-axis direction may be displayed on the scanogram image. The scanning condition display area 1001 displays a relational diagram showing a relationship between the scanning conditions and index values, i.e. the relational diagrams illustrated in FIGS. 9 and 10 for each position z in the body-axis direction of the object. Thus, by displaying the window 10, an operator can immediately check index values for each position in the body-axis direction.

### Third Embodiment

Next, a third embodiment will be described. In the second embodiment, a GUI (Graphical User Interface) is used to change a parameter on a peak of the plane FIG. 806, i.e. each parameter axis. In the present embodiment, parameters are changed by performing a mouse operation on sides of the plane FIG. 806.

That is, the present embodiment is different form the second embodiment in configuration for changing parameters, and the other components are similar to the second embodiment. Description of the similar components will be omitted.

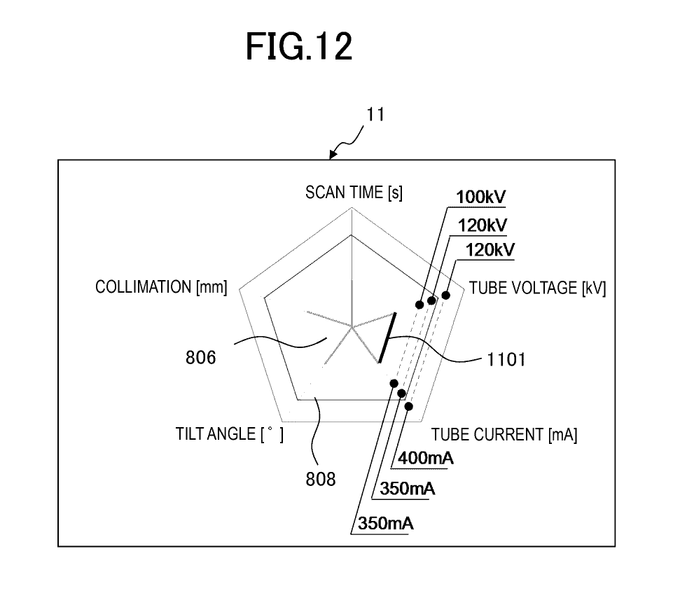

FIG. 12 shows an example of the relational diagram of the present embodiment. Although the configuration of the relational diagram 11 is similar to those of the relational diagrams 8 and 9 in FIGS. 9 and 10, a combination of the parameter values adjacent to each other across a side 1101 is set by performing a mouse operation on the side 1101 to specify the magnitudes of index values. For example, when the side 1101 is dragged on a side of the regular polygon 808 showing target values of the index values, candidates under a condition where the index values are equivalent to the target values are searched for a tube voltage and a tube current that are a combination of the parameters adjacent to each other across the side 1101, and the candidates are displayed on the relational diagram.

It is noted that “equivalent to the target values” means that values are within a predetermined range from the target values. In the relational diagram 11 of FIG. 12, 100 kV-350 mA, 120 kV-350 mA, and 120 kV-400 mA are displayed as a combination of a tube voltage and a tube current. An operator can select a desired scanning condition from among the displayed combinations. When candidates of a tube voltage and a tube current are searched, a scan time, a tilt angle, and a collimation that are parameters other than the tube voltage and the tube current are fixed at set values.

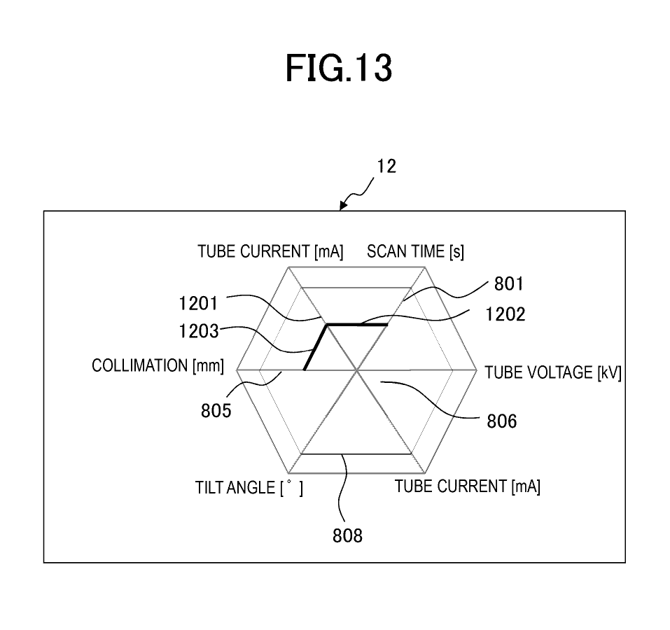

FIG. 3 shows the other example of the relational diagram of the present embodiment. In the relational diagram 11 of FIG. 12, a combination of parameters adjacent to each other across a side could be set by performing a mouse operation on a side of the plane FIG. 806. However, a combination of parameters that are not adjacent to each other, such as a combination of a tube current and a scan time, could not be set. Therefore, in the relational diagram 12 of FIG. 13, arrangement of the parameter axes can be changed, and a desired combination of parameters can be set.

In the relational diagram 12, a second axis 1201 of the tube current is disposed between the scan time axis 801 and the collimation axis 805. By thus disposing the second axis 1201 of the tube current, a combination of parameters that could not be set in the relational diagram 11 can be set. That is, a combination of the tube current and the scan time and a combination of the tube current and the collimation can be set by operating a side 1202 and a side 1203 respectively.

It is noted that a mouse operation is performed on an arc between two parameter axes instead of the side 1101 in a case where the plane FIG. 806 is not a regular polygon but a circle.

According to the embodiment described above, an index value can be estimated immediately by setting a parameter and the value from among scanning conditions, and candidates are searched for a desired combination of parameters, which can reduce a burden on an operator for setting scanning conditions.

### Fourth Embodiment

Next, a fourth embodiment will be described. An X-ray CT apparatus is taken as an example of a medical image diagnostic apparatus in the first to third embodiments. In the present embodiment, an MRI apparatus is taken as another example of the medical image diagnostic apparatus.

FIG. 13 is a schematic diagram of a configuration example of an MRI apparatus. An MRI apparatus 13 is provided with static magnetic field magnets 1302 that generate a static magnetic field around an object 1301, gradient magnetic field coils 1303 that generate a gradient magnetic field, irradiation coils 1304 that irradiate a high-frequency magnetic field pulse (referred to as “RF pulse”) to the object, reception coils 1305 that detect an NMR signal from the object, and a bed 1306 on which the object 1301 lies.

The static magnetic field magnets 1302 are disposed in a wide space around the object 1301, are made of any of permanent magnets; superconducting magnets; and normal conducting magnets, and generate a homogeneous static magnetic field in a direction parallel to or vertical to the body axis of the object 1301.

The gradient magnetic field coils 1303 apply gradient magnetic fields in the three axis directions X, Y, and Z to the object 1301 according to a signal from a gradient magnetic field power source 1307. According to the gradient magnetic field application method, a scanning cross section of the object is set.

The irradiation coils 1304 generate an RF pulse based on a signal of an RF transmission unit 1308. The RF pulse excites atomic nuclei of atoms composing biological tissues in the scanning cross section of the object 1301 set by the gradient magnetic field coils 1303, which induces an NMR (Nuclear Magnetic Resonance) phenomenon.

An echo signal, i.e. an NMR signal generated by the NMR phenomenon of the atomic nuclei of the atoms composing the biological tissues of the object 1301 that was induced by the RF pulse irradiated from the irradiation coils 1304, is detected by a signal detection unit 1309 through the reception coils 1305 disposed close to the object 1301, and signal processing is performed by a signal processing unit 1310 in order to be converted into an image. The converted image is displayed on a display unit 1311.

Parameters such as a repetition time (TR), an echo time (TE), and the like required for scanning are input to an input unit 1313 by an operator, and these parameters are transmitted and displayed on the display unit 1311. Similarly, these parameters are transmitted to a control unit 1312.

The control unit 1312 controls the gradient magnetic field power source 1307, the RF transmission unit 1308, and the signal processing unit 1310 in order to repeatedly generate an RF pulse and each of a slice encoding gradient magnetic field, a phase encoding gradient magnetic field, and a frequency encoding gradient magnetic field in a predetermined pulse sequence according to the parameters received from the input unit 1313.

A part of an RF pulse irradiated to the object 1301 is absorbed into the object 1301, which causes a negative effect such as body temperature rise. Therefore, in a case of scanning the object 1301 using the MRI apparatus, scanning conditions need to be set in consideration with an SAR (Specific Absorption Ratio) that is a ratio of the RF pulse to be absorbed into a human body. The SAR is proportional to a square of a static magnetic field, and it is required to pay attention to the SAR in particular when using a 3T high magnetic field device.

The control unit 1312 of the MRI apparatus of the present embodiment is provided with the index value calculation unit 20, the scale setting unit 21, and the display control unit 22 similarly to the first embodiment. These units work similarly to the first embodiment, which can lead to immediate understanding of a relationship between parameters of scanning conditions and an index values such as a SAR even in the MRI apparatus.

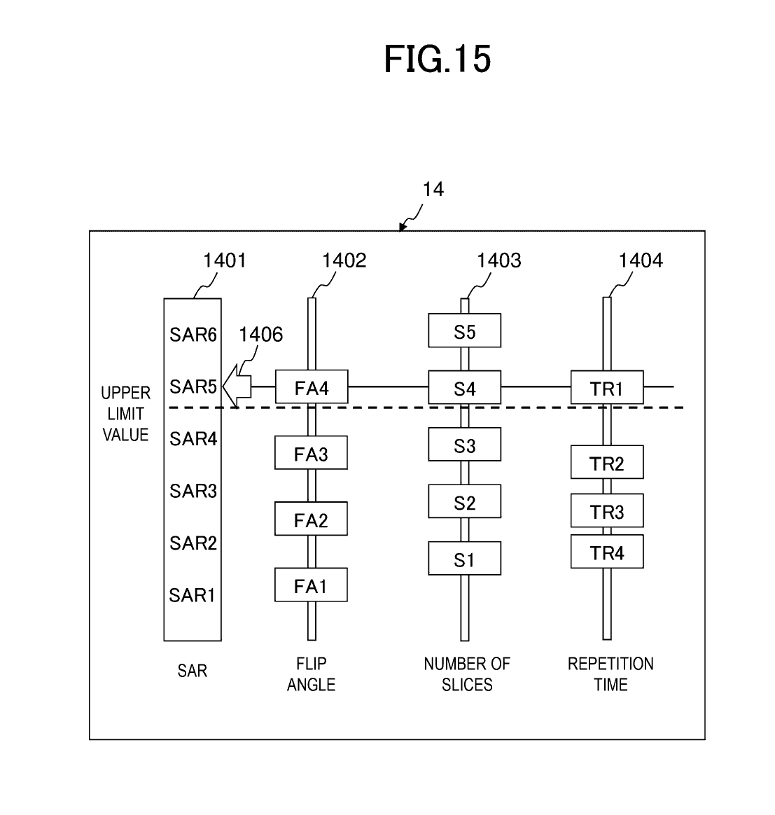

FIG. 15 shows an example relational diagram. A relational diagram 14 includes axes 1401 to 1404 and a marker 1406. The axes 1401 to 1404 represent a SAR, a flip angle, the number of slices, and a repetition time respectively and are arranged parallel to each other. Also, scales of the axes 1402 to 1404 respectively correspond to the index value axis 1401. That is, the axes correspond to the index value axis 1401 so that the index value can be estimated when one parameter is changed.

The marker 1406 indicates the magnitude of an index value calculated based on set scanning conditions. In the relational diagram 14 of FIG. 15, the tip of the marker 1406 indicates the magnitude of an index value when FA4, S4, and TR1 are set for a flip angle, the number of slices, and a repetition time respectively on the axis 1401. That is, this indicates that a SAR is set to SAR5.

Also, in the relational diagram 14, the upper limit value of the SAR is indicated as a SAR reference value by a broken line between D4 and D5. Displaying such a reference value helps an operator to determine whether or not the index value calculated based on scanning conditions is appropriate. In the relational diagram 14 of FIG. 15, it can be determined that the SAR exceeds the upper limit value under the set scanning conditions.

According to the embodiment described above, also in an MRI apparatus, an index value can be estimated immediately by setting a parameter and the value from among scanning conditions, which can reduce a burden on an operator for setting scanning conditions.

It is noted that the medical image display apparatus of the present invention is not limited to the above embodiments but can be embodied by transforming components within a scope that does not deviate from the gist of the invention.

Also, a plurality of components disclosed in the above embodiments may be combined as needed. Furthermore, some components may be removed from all the components shown in the above embodiments.

### REFERENCE SIGNS LIST

1: X-ray CT apparatus
100: scan gantry unit
101: X-ray tube device
102: rotating disk
103: collimator
104: opening
105: bed device
106: X-ray detector
107: data acquisition system
108: gantry controller
109: bed controller
110: X-ray controller
120: operation unit
121: input device
122: image processing device
123: storage device
124: system controller
125: display device
20: index value calculation unit
21: scale setting unit
22: display control unit
3: window
300: image display area
301: scanning condition display area
302: index value selecting part
4, 5, 6, and 7: relational diagrams
401: exposure dose axis
402: tube voltage axis
403: tube current axis
404: scan time axis
405: helical pitch axis
406: marker
701: image SD axis
8 and 9: relational diagrams
801: scan time axis
802: tube voltage axis
803: tube current axis
804: tilt angle axis
805: collimation axis
806: plane FIG.
807: tube voltage indication label
808: regular polygon showing target values of index values
10: window
1000: image display area
1001: scanning condition display area
11: relational diagram
1101: side
12: relational diagram
1201: second axis of the tube current
1202 and 1203: sides
1301: object
1302: static magnetic field magnets
1303: gradient magnetic field coils
1304: irradiation coils
1305: reception coils
1306: bed
1307: gradient magnetic field power source
1308: RF transmission unit
1309: signal detection unit
1310: signal processing unit
1311: display unit
1312: control unit
1313: input unit
14: relational diagram
1401: SAR axis
1402: flip angle axis
1403: axis of the number of slices
1404: repetition time axis
1406: marker

1. A medical image scanning apparatus that acquires and displays tomographic images of an object, comprising:
an index value calculation unit that calculates an index value based on each parameter value of scanning conditions;
a scale setting unit that sets a scale of each parameter axis according to the calculated index value; and
a display control unit that displays a relational diagram including a graphic indicating the magnitude of the calculated index value and each parameter axis having the set scale.

2. The medical image scanning apparatus according to claim 1,
wherein the graphic is a bar graph that indicates the magnitude of an index value or a marker that indicates the magnitude of the index value on the index value axis, and
the relational diagram is configured by arranging the bar graph or the index value axis, and each parameter axis parallel to each other.

3. The medical image scanning apparatus according to claim 1,
wherein the graphic is a plane figure that indicates the magnitudes of index values, and
the relational diagram is configured by arranging each parameter axis radially from the center of the plane figure.

4. The medical image scanning apparatus according to claim 3,
wherein an area of the plane figure corresponds to the magnitudes of the index values, and
scales of the respective parameter axes are set so as to correspond to square roots of the index values.

5. The medical image scanning apparatus according to claim 3,
wherein the relational diagram is displayed for each position in the body-axis direction of the object.

6. The medical image scanning apparatus according to claim 4,
wherein an image showing the position in the body-axis direction of the object is displayed with the relational diagram.

7. The medical image scanning apparatus according to claim 3,
wherein, when the magnitudes of the index values are specified by operating a side or an arc of the plane figure, and
candidates of scanning conditions for a combination of parameters adjacent to each other across the side or the arc are searched and displayed according to the magnitudes of the specified index values.

8. The medical image scanning apparatus according to claim 7,
wherein one of the parameter axes configuring the relational diagram is set as a second axis between the other parameter axes.

9. The medical image scanning apparatus according to claim 1,
wherein a reference value of the index value is displayed on the relational diagram.

10. A medical image scanning method that uses a medical image scanning apparatus acquiring and displaying tomographic images of an object, comprising:
a step of index value calculation that calculates an index value based on each parameter value of scanning conditions;
a step of scale setting that sets a scale of each parameter axis according to the calculated index value; and
a step of display control that displays a relational diagram including a graphic indicating the magnitude of a calculated index value and each parameter axis having the set scale.
